(19) United States
(12) Patent Application Publication
(10) Pub. No.: US 2025/0000970 A1
(43) Pub. Date: Jan. 2, 2025

(54) COMPOSITIONS AND METHODS FOR FOOD ALLERGY PREVENTION

(71) Applicant: Lil Mixins, LLC, Philadelphia, PA (US)

(72) Inventor: Meenal Lele, Philadelphia, PA (US)

(73) Assignee: Lil Mixins, LLC, Philadelphia, PA (US)

(21) Appl. No.: 18/845,942

(22) PCT Filed: Feb. 24, 2023
(86) PCT No.: PCT/US2023/013800

§ 371 Date: Sep. 11, 2024

Related U.S. Application Data
(60) Provisional application No. 63/313,956, filed on Feb. 25, 2022.

(51) Int. Cl. A61K 39/35; A23J 3/08; A23J 3/14; A23L 5/10; A23L 15/00; A23L 19/00; A23L 25/00; A61K 39/00

(52) U.S. Cl. CPC A61K 39/35; A23J 3/08; A23J 3/14; A23L 5/13; A23L 5/15; A23L 15/00; A23L 19/09; A23L 25/30; A61K 2039/542

(57) ABSTRACT

The instant disclosure provides compositions and methods useful for treating and/or preventing food allergy in a subject in need thereof. The compositions comprise allergenic proteins, wherein at least a portion of the allergenic proteins are natively folded and a portion are partially unfolded. The partially unfolded allergenic proteins comprises one or more T-cell epitopes and are substantially free of IgE epitopes and/or B-cell epitopes. Food compositions comprising a mix of naturally folded and T-cell epitope containing proteins can optimally balance reduced immunogenicity with adequate antigenic properties. Furthermore, by preserving the food in as natural form as possible, they induce an overall tolerance training effect.

### FIELD

The instant application is generally directed to compositions and methods for altering the allergic response to a food protein in a subject in need thereof.

### BACKGROUND

Food allergies and other types of food hypersensitivities occur when the body's immune system reacts to certain proteins in food. Food allergic reactions vary in severity from mild symptoms involving hives and lip swelling to severe, life-threatening symptoms, often called anaphylaxis, that may involve fatal respiratory problems and shock. The cause of food allergies is not well understood. While promising prevention and therapeutic strategies are being evaluated, food allergies currently cannot be cured.

Accordingly, there exists a need for a new class of food proteins with an improved safety profile.

### BRIEF SUMMARY

Provided herein are compositions and methods for treating and/or preventing food allergy in a subject in need thereof. The compositions comprise allergenic proteins, wherein at least a portion of the allergenic proteins are natively folded and a portion are partially unfolded.

In some embodiments, the disclosure provides a food composition comprising an allergenic protein; wherein between 1% and 99% of the allergenic protein in the composition is partially unfolded, wherein the partially unfolded allergenic protein comprises one or more T-cell epitopes and is substantially free of IgE epitopes and/or B-cell epitopes; and wherein at least 1% of the allergenic protein in the composition is natively folded. In some embodiments, between 1% and 25%, between 25% and 50%, between 50% and 75%, or between 75% and 99% of the allergenic protein is partially unfolded.

In some embodiments, the allergenic protein is from peanut, egg, milk, wheat, almond, cashew, pistachio, hazelnut, walnut, pecan, brazil nut, soy, oat, pea, cod, salmon, shrimp, or sesame. In some embodiments, the allergenic protein is from peanut and is Ara h 1, Ara h 2, Ara h 3, Ara h 6, or Ara h 9. In some embodiments, the allergenic protein is from egg and is Gal d 1, Gal d 2, Gal d 3, Gal d 4, or Gal d 5. In some embodiments, the allergenic protein is from milk and is Bos d 5 or Bos d 11. In some embodiments, the allergenic protein is from walnut and is Jug r1 or Jug r3. In some embodiments, the allergenic protein is from pistachio and is Pis v 1, Pis v 2, Pis v 3, or Pis V 4. In some embodiments, the allergenic protein is from cashew and is AnaO3. In some embodiments, the allergenic protein is from hazelnut and is Cor a 9, Cor a 14, Cor a 1, or Cor a 8. In some embodiments, the allergenic protein is from pecan and is Car i 1. In some embodiments, the allergenic protein is from almond and is Pru du 1, 2, 3, 4, 5, 6, or 8. In some embodiments, the allergenic protein is from brazil nut and is Ber e1 or e2. In some embodiments, the allergenic protein is from wheat and is Tri a 19 or Tri a 14. In some embodiments, the allergenic protein is from soy and is Gly m Bd 60K, Gly m Bd 30K, or Gly m Bd 28K. In some embodiments, the allergenic protein is from pea and is Pis s 1 or Pis s 2. In some embodiments, the allergenic protein is from cod and is Gad m 1, Gad m 2, or Gad m 3. In some embodiments, the allergenic protein is from salmon and is Sal s 1, Sal s 3, Sal s 2, or Onc k 5. In some embodiments, the allergenic protein is from shrimp and is Met e 1, Pan s 1, or Hom a 1. In some embodiments, the allergenic protein is from oat and is avenin.

In some embodiments, the food composition comprises one or more fats, fibers, and/or vitamins. In some embodiments, the food composition comprises butyrate. In some embodiments, the food composition comprises one or more non-allergenic proteins. In some embodiments, the food composition comprises at least one probiotic, such as lactobacillus or bifidobacterium. In some embodiments, the food composition comprises a prebiotic, such as an oligosaccharide.

In some embodiments, the food composition is a powder, a puree, a paste, or a beverage. In some embodiments, the food composition is a puff, cracker, or biscuit.

Also provided is a method of partially unfolding an allergenic protein in a food composition, the method comprising treating the food composition using: i) heat treatment; ii) instant controlled pressure drop; iii) pepsination; iv) hydrolyzation; v) acid or base treatment and/or vi) fermentation; wherein the treatment reduces the amount of natively folded allergenic protein in the food composition by at least 50%; and wherein the partially unfolded allergenic protein comprises one or more T-cell epitopes and is substantially free of IgE epitopes and/or B-cell epitopes. In some embodiments, the treatment reduces the amount of natively folded allergenic protein in the food composition by at least 75%, at least 90%, at least 95%, or at least 99%. In some embodiments, between 1% and 99% of the allergenic protein in the food composition is partially unfolded after the treatment.

In some embodiments, the food composition is freeze dried. In some embodiments, the food composition is a wet composition that is mixed with a starch before treatment.

In some embodiments, the heat treatment comprises boiling or treatment with super heated steam. In some embodiments, the boiling or treatment with steam is for about 1-5, about 5-10, about 10-20, about 20-30, or more than 30 minutes. In some embodiments, the heat treatment comprises baking or toasting. The baking or toasting may be, in some embodiments, for about 1-5, about 5-10, about 10-20, about 20-30, or more than 30 minutes.

Also provided is a method of treating and/or preventing food allergy in a subject in need thereof, the method comprising administering to the subject a food composition of the disclosure. In some embodiments, the subject is a pediatric subject. In some embodiments, the subject is 18 months of age or less. In some embodiments, the subject is 12 months of age or less. In some embodiments, the subject is 6 months of age or less. In some embodiments, the food composition is administered orally.

Also provided is a kit comprising a vessel comprising a food composition described herein. In some embodiments, the vessel is a sachet, bag, or tube. In some embodiments, the vessel is a spoon. In some embodiments, the vessel comprises a single dose of the food composition. In some embodiments, the vessel comprises multiple doses of the food composition. In some embodiments, the vessel further comprises a second food composition comprising a second allergenic protein. In some embodiments, the kit comprises a second vessel. In some embodiments, the second vessel comprises a second allergenic protein.

In some embodiments, the kit comprises a first vessel and second vessel, wherein the amount of partially denatured allergenic protein in the first vessel is different from the amount of partially denatured allergenic protein in the second vessel. In some embodiments, the kit comprises a first vessel and second vessel, wherein the amount of partially denatured allergenic protein in the first vessel is the same as the amount of partially denatured allergenic protein in the second vessel. In some embodiments, the kit comprises a first, second, third, and fourth vessel, wherein the amount of partially denatured allergenic protein is different in each vessel. In some embodiments, the kit comprises a first, second, third, and fourth vessel, wherein the amount of partially denatured allergenic protein is the same in each vessel.

These and other features, aspects and advantages of the present invention will become better understood with regard to the following description, appended claims, examples and accompanying figures.

### BRIEF DESCRIPTION OF THE DRAWINGS

Various objects and advantages and a more complete understanding of the disclosure are apparent and more readily appreciated by reference to the following Detailed Description and to the appended claims when taken in conjunction with the accompanying Drawings, wherein:

FIG. 1 is a schematic showing modification of a natural food composition (left of the arrow) to produce a composition comprising a mixture of natively-folded and partially unfolded proteins.

FIGS. 2A-2B show levels of natively-folded Gal d 1, 2 (FIG. 2A) and Bos d 5, 11 (FIG. 2B) after toasting for 5 or 10 minutes.

FIGS. 3A-3B show levels of natively-folded Gal d 1, 2 (FIG. 3A) and Bos d 5, 11 (FIG. 3B) after boiling for 20, 30, or 30 min.

FIG. 4 shows levels of natively-folded Gal d 1, 2 after baking for 10, 20, or 30 minutes.

FIGS. 5A-5B show levels of natively-folded Gal d 1, 2 (FIG. 5A) and Bos d 5, 11 (FIG. 5B) after boiling for 10 mins followed by toasting for 5 or 10 minutes.

FIG. 6 provides an “x” selection point of the high and low ends of a percent range of broken weak bonds relative to all of the weak bonds in a given allergenic protein.

FIG. 7 provides a “y” selection point of the high and low ends of a percent range of the amount by weight of a given allergenic protein relative to the total weight of all protein(s) in a food composition.

FIG. 8 shows the effect of boiling time on peanut protein structure at acidic pH (pH 3.04 initially) by circular dichromism.

FIG. 9 shows the effect of boiling time on peanut protein structure at neutral pH (pH 7 initially) by circular dichromism.

FIG. 10 shows the effect of boiling time on peanut protein structure at basic pH (pH 8.02 initially) by circular dichromism.

FIG. 11 shows effect of treatment on the allergenicity of the protein Ara h1 (Acidic=pH˜3; Basic=pH˜8).

FIG. 12 shows effect of treatment on the allergenicity of the protein Ara h3 (Acidic=pH˜3; Basic=pH˜8).

FIG. 13 shows effect of treatment on the allergenicity of the protein Ara h6 (Acidic=pH˜3; Basic=pH˜8).

FIG. 14 shows the effect of boiling time on almond protein structure at acidic pH (pH 3.04 initially) by circular dichromism.

FIG. 15 shows the effect of boiling time on almond protein structure at neutral pH (pH 7 initially) by circular dichromism.

FIG. 16 shows the effect of boiling time on almond protein structure at basic pH (pH 8.02 initially) by circular dichromism.

FIG. 17 shows effect of treatment on the allergenicity of the protein Pru du 6 (Acidic=pH˜3; Basic=pH˜8).

### DETAILED DESCRIPTION

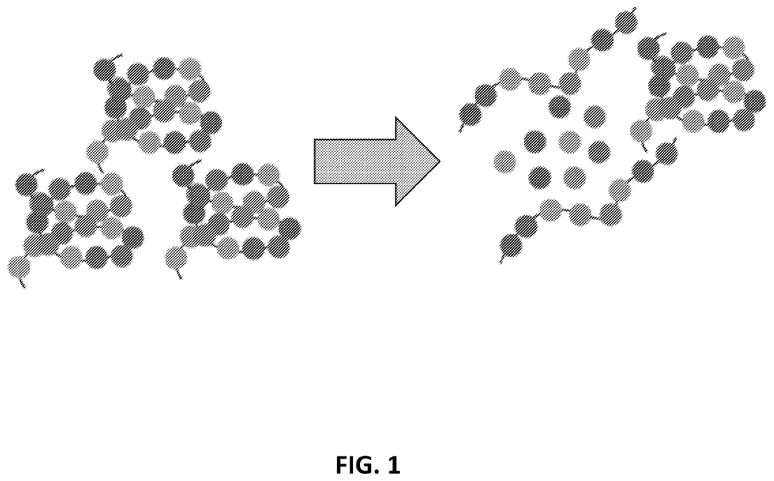

The instant disclosure provides compositions and methods useful for treating and/or preventing food allergy in a subject in need thereof. The compositions comprise allergenic proteins, wherein at least a portion of the allergenic proteins are natively folded and a portion are partially unfolded. Food compositions containing a mix of naturally folded and partially unfolded proteins, as shown in FIG. 1, can optimally balance reduced immunogenicity with adequate antigenic properties. Furthermore, by preserving the food in as natural form as possible, they induce an overall tolerance training effect.

Use of compositions comprising a mixture of partially unfolded and natively folded allergenic proteins provides distinct advantages over methods for preventing and/or treating food allergies known in the art. The only current method for food allergy prevention, known as “early allergen introduction,” uses compositions comprising only natively folded (i.e., wildtype) allergenic proteins, which provoke a large immunogenic response. Such compositions lead to high IgE activation. The use of isolated hypoallergenic proteins known in the art, which have very low levels of IgE activation, is less desirable for preventing and/or treating food allergies because these proteins do not provide the context (i.e., fat, fiber, nutrients) in which persons will naturally encounter proteins. T-cell epitope-containing peptides lack IgE and IgG epitopes, and also do not provide the natural context. In contrast, the compositions provided herein comprise partially unfolded allergenic proteins that comprise one or more T-cell epitopes and IgG epitopes, and are substantially free of IgE epitopes and/or B-cell epitopes. Without being bound by any theory, it is believed that the fact these proteins substantially lack IgE epitopes and/or B-cell epitopes but retain T-cell and IgG epitopes, and retain the natural protein context, helps induce preventative and/or therapeutic immune tolerance thereof. Thus, the disclosed compositions have a reduced immunogenic profile compared to known food supplements in the art and have an antigenic or tolerogenic profile that more safely and effectively prevents and/or treats food allergies.

The present invention is described more fully hereinafter using illustrative, non-limiting embodiments, and references to the accompanying figures. This invention may, however, be embodied in many different forms and should not be construed as to be limited to the embodiments set forth below. Rather, these embodiments are provided so that this disclosure is thorough and conveys the scope of the invention to those skilled in the art.

### Definitions

Unless otherwise defined in the disclosure, scientific and technical terms used in this application shall have the meanings that are commonly understood by those of ordinary skill in the art. Generally, nomenclature used in connection with, and techniques of, food science, chemistry, molecular biology, cell biology, immunology, microbiology, pharmacology, and protein and nucleic acid chemistry, described in the disclosure, are those well-known and commonly used in the art.

As used in the disclosure, the following terms have the meanings ascribed to them unless specified otherwise.

The articles “a,” “an,” and “the” are used in the disclosure to refer to one or to more than one (i.e., to at least one) of the grammatical object of the article. By way of example, “an element” means one element or more than one element.

The use of the alternative (e.g., “or”) should be understood to mean either one, both, and any combination thereof of the alternatives.

The term “and/or” should be understood to mean either one, or both of the alternatives.

As used in the disclosure, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In some embodiments, the term “about” or “approximately” refers a range of quantity, level, value, number, frequency, percentage, dimension, size, amount, weight or length±15%, ±10%, +9%, ±^{8%}, 7%, ±6%, 5%, ±4%, ±3%, ±2%, or ±1% about a reference quantity, level, value, number, frequency, percentage, dimension, size, amount, weight or length.

As used in the disclosure, the term “isolated” means material that is substantially or essentially free from components that normally accompany it in its native state. In some embodiments, the term “obtained” or “derived” is used synonymously with isolated.

A “subject,” “individual,” or “patient” as used herein, includes any animal that may ingest or use compositions contemplated herein. Suitable subjects include laboratory animals (such as mouse, rat, rabbit, or guinea pig), farm animals (such as horses, cows, sheep, pigs), and domestic animals or pets (such as a cat or dog). In some embodiments, the subject is a mammal. In some embodiments, the subject is a non-human primate. In some embodiments, the subject is a human. In some embodiments, the subject is an adult human. In some embodiments, the subject is a pediatric human. In some embodiments, a pediatric subject is 18 months of age or less. In some embodiments, a pediatric subject is 12 months of age or less. In some embodiments, a pediatric subject is 6 months of age or less.

As used herein, the term “allergenic protein” refers to a protein capable of eliciting an allergic response in a subject that is sensitive thereto. An allergic response is a vigorous immune response in which the immune system fights off a perceived threat that would otherwise be harmless to the body. The allergic response may be characterized by production of antibodies, including IgE antibodies, against the allergenic protein. Symptoms of an allergic response may include allergic rhinitis (e.g., sneezing and itchy, runny or blocked nose); conjunctivitis (e.g., itchy, red, and/or watering eyes); wheezing, chest tightness, shortness of breath and/or cough; hives and/or rash; swelling of the lips, tongue, eyes or face; stomach pain, vomiting, and/or diarrhea; dry, red and/or cracked skin.

As used herein, the term “natively folded” refers to a protein in its properly folded and/or assembled form, which is operative and functional. Natively folded allergenic proteins comprise one or more IgE epitopes and/or B-cell epitopes, which may elicit an immune response in a subject. In contrast, the term “partially unfolded” refers to a protein in which the tertiary and/or secondary structure is altered compared to a natively folded state. Partially unfolded proteins may have a reduced number of IgE epitopes and/or B-cell epitopes compared to their natively folded counterparts. In some embodiments, a partially unfolded protein may be free or substantially or completely free of IgE epitopes and/or B-cell epitopes.

As used herein, the term “IgE epitope” refers to an epitope present on an allergenic protein that is recognized by IgE antibodies produced by a subject. In some embodiments, IgE epitopes are discontinuous epitopes, such as conformational epitopes.

As used herein, the term “B-cell epitope” refers to an epitope present on an allergenic protein that is recognized by B-cells produced by a subject. In some embodiments, B-cell epitopes are discontinuous epitopes, such as conformational epitopes.

### Allergenic Proteins

Allergenic proteins can be found in many different types of foods, such as nuts and dairy products. For example, in some embodiments, an allergenic protein is from a natural source. Natural sources can include one or more of peanut, egg, milk, wheat, almond, cashew, pistachio, hazelnut, walnut, pecan, brazil nut, soy, oat, pea, cod, salmon, shrimp, or sesame. A non-limiting list of allergenic proteins is provided in Table 1.

TABLE 1
Illustrative Allergenic Proteins

| Food Source | Allergenic Proteins |
|---|---|
| Peanut | Ara h 1, Ara h 2, Ara h 3, Ara h 6, Ara h 9 |
| Egg | Gal d 1, Gal d 2, Gal d 3, Gal d 4, Gal d 5 |
| Milk | Bos d 5 or Bos d 11 |
| Walnut | Jug r1, Jug r3 |
| Pistachio | Pis v 1, Pis v 2, Pis v 3, Pis V 4 |
| Cashew | AnaO3 |
| Hazelnut | Cor a 9, Cor a 14, Cor a 1, Cor a 8 |
| Pecan | Car i 1 |
| Almond | Pru du 1, Pru du 2, Pru du 3, Pru du 4, Pru du 5, Pru du 6, Pru du |
| | 8 |
| Brazil nut | Ber e1, Ber e2 |
| Wheat | Tri a 19, Tri a 14 |
| Soy | Gly m Bd 60K, Gly m Bd 30K, Gly m Bd 28K |
| Pea | Pis s 1, Pis s 2 |
| Cod | Gad m 1, Gad m 2, Gad m 3 |
| Salmon | Sal s 1, Sal s 3, Sal s 2, Onc k 5 |
| Shrimp | Met e 1, Pan s 1, or Hom a 1 |
| Oat | Avenin |

As described herein, an allergenic protein may be in a naively folded state. A natively folded allergenic protein may comprise one or more IgE epitopes and/or B-cell epitopes and may be capable of eliciting an allergic response in a subject that is sensitive thereto. In some embodiments, a natively folded allergenic protein comprises one or more T-cell epitopes. In some embodiments, a natively folded allergenic protein comprises one or more IgG antibody epitopes.

In some embodiments, an allergenic protein may be in a partially unfolded state. A partially unfolded protein may comprise a reduced number of IgE epitopes and/or B-cell epitopes as compared to the same protein in a natively folded state. For example, partially unfolding a protein may reduce the number of IgE epitopes and/or B-cell epitopes in the protein as compared to the natively folded state by at least 10%, at least 20%, at least 30%, at least 40%, at least 50%, at least 60%, at least 70%, at least 80%, or at least 90%, at least 95%, a least 96%, at least 97%, at least 98%, or at least 99%. In some embodiments, partially unfolding a protein may eliminate the number of IgE epitopes and/or B-cell epitopes in a protein as compared to the natively folded state. In some embodiments, partially unfolding a protein may eliminate the number of B-cell epitopes in a protein as compared to the natively folded state. In some embodiments, partially unfolding a protein may reduce, but not eliminate the number of T-cell epitopes in a protein, as compared to the natively folded state. In some embodiments, partially unfolding a protein may not substantially reduce the number of T-cell epitopes in a protein, as compared to the natively folded state. In some embodiments, partially unfolding a protein may reduce, but not eliminate, the number of IgG antibody epitopes in a protein, as compared to the natively folded state. In some embodiments, partially unfolding a protein may not substantially reduce the number of IgG antibody epitopes in a protein, as compared to the natively folded state.

In some embodiments, any of the (a) percent unfolding, (b) percent change in conformational structure, (c) percent change in T-cell epitopes, (d) percent change of IgE epitopes and/or B-cell epitopes in the protein, or (e) percent reduction or freedom of IgE epitopes and/or B-cell epitopes, as compared to the native structural state, can be: 1%, 2%, 3%, 4%, 5%, 6%, 7%, 8%, 9%, 10%, 11%, 12%, 13%, 14%, 15%, 16%, 1^{7%}, 18%, 9%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70, 71%, 72%, 73%, 74%, 75%, 76%, 77%, 78%, 79%, 80%, 81%, 82%, 83%, 84%, 85%, 86%, 87%, 88%, 89%0, 90%, 91%, 92%, 93%, 94%, 95%, 96%, 97%, 98%, 99%, and 100%

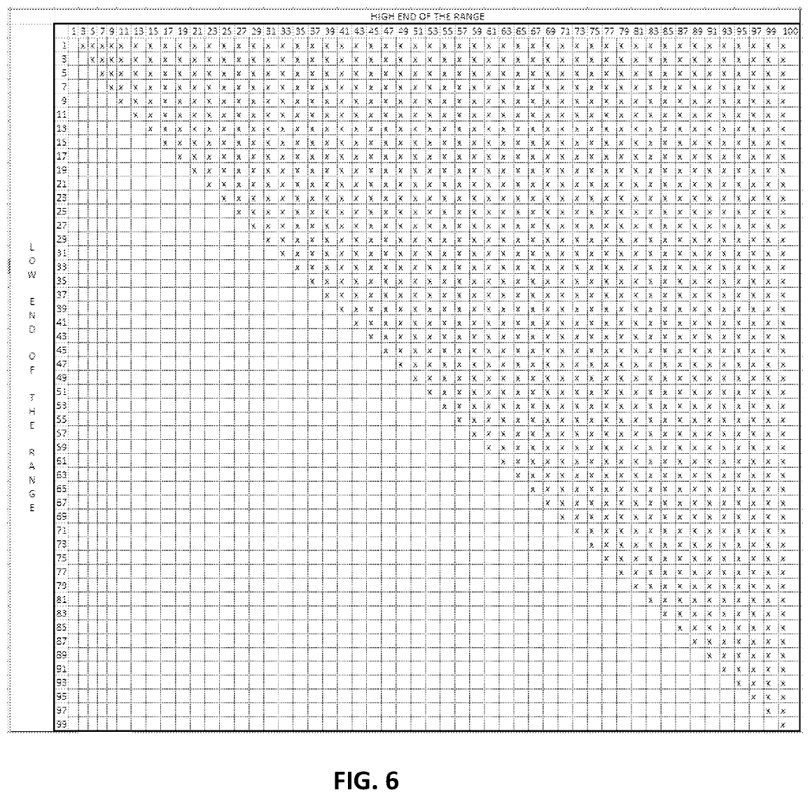

FIG. 6 provides for some embodiments details concerning the selection of a range specifying the degree of weak bonds permanently broken compared to all of the weak bonds in a given allergenic protein (e.g., an allergenic protein selected from the rightmost column in Table 1) relative to that allergenic protein's natively folded state. In particular, FIG. 6 provides an “x” selection point that establishes the high and low ends of a percent range of broken weak bonds (which correlates to a degree of unfolding or a reduced number of epitopes of a given protein) relative to all of the weak bonds of the given allergenic protein in its natively folded state.

### Methods of Partially Unfolding Proteins

Also provided herein are methods for partially unfolding proteins, such as allergenic proteins, and methods for producing food compositions comprising partially unfolded proteins. In certain embodiments, a portion (e.g., an unfolded portion) of an allergenic protein includes one or more T-cell epitopes and is substantially free of IgE epitopes and/or B-cell epitopes

The methods described herein allow for partial unfolding of proteins, to a known and controllable extent. In some embodiments, the methods described herein allow for partial unfolding of a protein, such that between 1% and 99% of an allergenic protein is unfolded. For example, using the methods described herein, between about 1% and about 25%, about 25% and about 50%, about 50% and about 75%, about 75 and about 99% of the allergenic protein is unfolded.

Illustrative methods for partially unfolding an allergenic protein include, but are not limited to, heat treatment, instant controlled pressure drop, pepsination, hydrolyzation, acid treatment or fermentation. Other methods known to those of skill in the art for partially unfolding proteins may also be used. Treatment with one or more of these methods may reduce the amount of natively folded allergenic protein, may reduce or eliminate IgE epitopes and/or B-cell epitopes, and/or substantially maintain one or more T-cell epitopes on the food composition.

As used herein “heat treatment” may include any methods to apply heat to natively-folded protein without chemically transforming (i.e., burning) the protein. Illustrative heat treatment methods may include, for example, wet heat methods (e.g., boiling, or steam) or dry heat methods (e.g., baking or toasting). During heat treatment, the temperature may stay constant or may vary over time. In some embodiments, heat treatment comprises boiling or treatment with steam. The boiling or treatment with steam may be for about 1-5, about 5-10, about 10-20, about 20-30, or more than 30 minutes. In some embodiments, the heat treatment comprises baking or toasting. The baking or toasting may be for about 1-5, about 5-10, about 10-20, about 20-30, or more than 30 minutes.

As used herein “instant controlled pressure drop” refers to a process comprising a short heating step (e.g., heating for about 10-60 seconds), including a saturated steam or pressure air injection under high-pressure (e.g., up to 1 MPa) applied to a natively-folded protein put initially under a vacuum. Following the first heating step, the abrupt dropping of pressure (e.g., about 0.5 MPa/sec) toward a vacuum (e.g., about 3-5 kPa) over only a short time (e.g., about 10-60 milliseconds) results in significant mechanical stress enabling the product to be expanded. The pressure drop also allows rapid cooling, which prevents the thermal degradation of sensitive compounds in the composition, such as compounds which contribute to one or more organoleptic properties such as taste.

Pepsination refers to the use of the pepsin enzyme to partially unfold a natively-folded protein. Pepsin is an endopeptidase that breaks down dietary proteins in the stomach into amino acids that can be easily absorbed in the small intestine. It functions by digesting peptide or hydrogen (weak) bonds, the predominant chemical bonds found in proteins that enable complex structures. Such bonds are often IgE binding sites/comprised in IgE epitopes and/or B-cell epitopes. Partial unfolding of a protein using a pepsination approach may be controlled by varying concentration of enzyme used, and/or time of treatment.

Hydrolyzation refers to a chemical reaction in which a molecule of water breaks one or more peptide bonds in a wild-type folded protein. Typically, this process is used to break proteins into peptide chains or amino acids, but may be controlled to reduce the number of IgE binding sites.

Acid or base treatment involves the use of one or more acids of bases to break peptide or hydrogen bonds. As noted above, peptide bonds are responsible for the secondary or tertiary structure of proteins. Destruction of a bond may eliminate one or more IgE epitopes and/or B-cell epitopes in a protein. Acid or base treatments described herein may employ weaker or stronger acids or bases, in order to control the extent of protein unfolding. Time of treatment may also be varied.

Fermentation involves the chemical breakdown of a protein peptide or hydrogen bonds by bacteria, yeasts, or other microorganisms. Fermentation described herein may employ one or more organisms, in sequence or concurrently in order to control the protein changes. Time of fermentation, and addition of heat or cooling may also be varied.

In some embodiments, a method of partially unfolding an allergenic protein involves treating an isolated protein in accordance with the above, and then creating a food composition that includes the unfolded protein.

Alternatively, it is contemplated that directly treating at least a portion of a food composition to at least partially unfold an allergenic protein in the food composition portion, can be used. In such embodiments, suitable techniques typically include treating the food composition with a combination of two or more of heat treatment, instant controlled pressure drop, pepsination, hydrolyzation, acid treatment, or fermentation.

The food composition to which the treatment is applied may be in many different forms. For example, in some embodiments, the food composition is freeze dried. In some embodiments, the food composition is a dry composition. In some embodiments, the food composition is a wet composition. Wet compositions may be, in some embodiments, mixed with a starch before treatment. Illustrative starches that may be used in the instant methods include, but are not limited to, tapioca starch, cassava flour, maize starch, wheat starch, rice starch, or potato starch.

In some embodiments, a method of partially unfolding an allergenic protein in a food composition comprises treating the food composition using heat treatment, instant controlled pressure drop, pepsination, hydrolyzation, acid or base treatment, or fermentation; wherein the treatment reduces the amount of natively folded allergenic protein in the food composition by at least 50%; and wherein the partially unfolded allergenic protein comprises one or more T-cell epitopes and/or IgG epitopes and is substantially free of IgE epitopes and/or B-cell epitopes. In some embodiments, the treatment reduces the amount of natively folded allergenic protein in the food composition by at least 75%. In some embodiments, the treatment reduces the amount of natively folded allergenic food protein in the food composition by at least 90%. In some embodiments, the treatment reduces the amount of natively folded allergenic food protein in the food composition by at least 95%. In some embodiments, the treatment reduces the amount of natively folded allergenic food protein in the food composition by at least 99%. In some embodiments, between 1% and 99% of the allergenic protein in the food composition is partially unfolded after the treatment, such as about 1% to about 25%, about 25% to about 50%, about 50% to about 75%, or about 75% to about 99%.

In some examples, the method of partially unfolding an allergenic protein in a food composition comprises treating the food composition using heat treatment, instant controlled pressure drop, pepsination, hydrolyzation, acid treatment, or fermentation; wherein the treatment further comprises an acidic pH, a neutral pH or a basic pH. An acidic pH may be less than 7, less than 6, less than 5, less than 4, less than 3, less than 2, or less than 1. A neutral pH may be about 7. A basic pH may be more than 7, more than 8, more than 9, more than 10, more than 11, more than 12, or more than 13.

Methods of Evaluating the Degree of Unfolding and/or Change or Reduction of the Number of IgE epitopes and/or B-cell epitopes in the Protein as Compared to the Natively Folded State of a Given Allergenic Protein

Controlling, using the techniques described herein for example, and evaluating the degree of unfolding, and also correlating the degree of unfolding to a differential number of epitopes (e.g., percent IgE epitopes and/or B-cell epitopes relative to a natively folded state) of a given protein, is generally understood. It is generally assumed that there is a direct relationship between antibody accessibility to antigens and accessible surface of proteins. Based on this assumption, prediction systems often include solvent accessibility values calculated from the primary sequence of proteins or from their three-dimensional structures as a predictive criterion.

Techniques to evaluate the degree of unfolding of a protein include solubility analysis, e.g., the more insoluble the protein is in a given solvent the greater the degree of permanently broken weak bond and unfolding can be determined. Other techniques, including light scatter, SEM, circular dichroism, and pH shift can also be used to quantify a protein's shift away from a natively folded state.

In one embodiment, the difference in folding and/or degree of T-cell epitopes can be determined by T-cell 1 binding in sera.

In one embodiment, the difference in folding and/or degree of IgE epitopes and/or B-cell epitopes can be determined by IgE binding binding in sera or with monoclonal antibodies.

In one embodiment, the difference in folding and/or degree of B-cell epitopes can be determined by B-cell binding binding in sera.

The methods employed and the units used normally measure the allergen content and biological activity. Examples hereof are SQ-Units (Standardised Quality units), BAU (Biological Allergen Units), BU (biological units), UM (Units of Mass), IU (International Units) and IR (Index of Reactivity). Further, guidance to the normally applied, acceptable tests measuring biopotency of allergens are found e.g. in Note for Guidance on Allergen Product; The European Agency for the Evaluation of Medicinal Product, CPMP_BWP-243-96, London, 1996.

Several laboratory tests are also available for characterizing an allergen. The most widely used techniques are sodium dodecyl sulfate polyacrylamide gel electrophoresis (SDS-PAGE), isoelectric focusing (IEF), crossed immunoelectrophoresis (CIE) and Rocket Immuno Electrophoresis (RIE). The quantification of individual allergens may be performed by a variety of quantitative immunoelectrophoretic techniques (QE), Radial Immune Diffusion (RIE) or by enzyme-linked immunosorbent assays (ELISA). The determination of total allergenic activity is most frequently performed by radio allergosorbent test (RAST), Magic Lite assay (LIA) or related techniques. ELISA-based techniques may also be used.

Techniques for evaluation of protein allergens is detailed in US Publication No. 20110200641, the entire content of which is incorporated by reference as if fully set forth herein,

### Food Compositions

Provided herein are food compositions for treating and/or preventing food allergy in a subject in need thereof. The compositions comprise at least one allergenic protein, wherein at least a portion of the allergenic protein is partially unfolded, as detailed above, and optionally one or more additional constituents, such as fat, fiber, vitamins, minerals, and carbohydrates.

In some embodiments, food composition comprises an allergenic protein. The amount by weight of the allergenic protein in the food composition is detailed further below. If included in a food composition, between 1% and 99% of the allergenic protein in the composition is partially unfolded. In some embodiments, between 1% and 25%, between 25% and 50%, between 50% and 75%, or between 75% and 99% of the allergenic protein is partially unfolded. Other ranges and values for a degree of unfolding are detailed above.

In some embodiments, a food composition comprises a partially unfolded allergenic protein that comprises one or more T-cell epitopes and IgG epitopes. The presence of T-cell epitopes and IgG epitopes in a partially unfolded allergenic protein may promote tolerance thereof by a subject's immune system, i.e., the presence of T-cell epitopes and/or IgG epitopes may prevent the development of an allergic response to a protein.

In some embodiments, a food composition comprises a partially unfolded protein that is substantially free of IgE epitopes and/or B-cell epitopes. The absence of an IgE epitope and/or B-cell epitope in a partially unfolded protein may promote tolerance thereof by a subject's immune system, i.e., the absence of an IgE and/or B-cell epitope may prevent the development of an allergic response to a protein.

In some embodiments, one or more portions of a food composition comprises an unfolded allergenic protein and the remainder of the protein comprises natively folded allergenic protein. Basic subtraction of the amount by weight of the partially or fully unfolded proteins present in a food composition from the overall amount of protein in the food composition, permits calculation of the amount of protein that remains in its native state. In some embodiments, at least 1%, by weight, of the allergenic protein in the composition is natively folded. For example, in some embodiments, between 1% and 25%, between 25% and 50%, between 50% and 75%, or between 75% and 99%, by weight, of the allergenic protein is natively folded.

Without being bound by any theory, it is believed that a food composition comprising both natively folded and partially unfolded forms of the same protein may promote tolerance of the protein by a subject's immune system. This effect is greater when a subject is exposed to a mixture of natively folded and partially unfolded forms, as opposed to exposure to only partially unfolded forms.

The allergenic protein of the food composition may be isolated or derived from, for example, peanut, egg, milk, wheat, almond, cashew, pistachio, hazelnut, walnut, pecan, brazil nut, soy, oat, pea, cod, salmon, shrimp, or sesame. In some embodiments, the allergenic protein is any one of the proteins listed in Table 1. In some embodiments, a food composition may comprise two or more allergenic proteins, such as three, four, five, six, seven, eight, nine, ten or more allergenic proteins. In embodiments wherein the food composition comprises two or more allergenic proteins, each of the allergenic proteins may be selected from those listed in Table 1.

In some embodiments, a food composition comprises an allergenic protein selected from Ara h 1, Ara h 2, Ara h 3, Ara h 6, and Ara h 9. In some embodiments, a food composition comprises an allergenic protein selected from Gal d 1, Gal d 2, Gal d 3, Gal d 4, and Gal d 5. In some embodiments, a food composition comprises an allergenic protein selected from Bos d 5 and Bos d 11. In some embodiments, a food composition comprises an allergenic protein selected from Jug r1 and Jug r3. In some embodiments, a food composition comprises an allergenic protein selected from Pis v 1, Pis v 2, Pis v 3, and Pis V 4. In some embodiments, a food composition comprises an allergenic protein, wherein the allergenic protein is AnaO3. In some embodiments, a food composition comprises an allergenic protein selected from Cor a 9, Cor a 14, Cor a 1, and Cor a 8. In some embodiments, a food composition comprises an allergenic protein, wherein the allergenic protein is Car i 1. In some embodiments, a food composition comprises an allergenic protein, wherein the allergenic protein is selected from Pru du 1, Pru du 2, Pru du 3, Pru du 4, Pru du 5, Pru du 6, and Pru du 8. In some embodiments, a food composition comprises an allergenic protein, wherein the allergenic protein selected from Ber e1 or e2. In some embodiments, a food composition comprises an allergenic protein, wherein the allergenic protein is selected from Tri a 19 and Tri a 14. In some embodiments, a food composition comprises an allergenic protein, wherein the allergenic protein is selected from Gly m Bd 60K, Gly m Bd 30K, and Gly m Bd 28K. In some embodiments, a food composition comprises an allergenic protein, wherein the allergenic protein is selected from Pis s 1 and Pis s 2. In some embodiments, a food composition comprises an allergenic protein, wherein the allergenic protein is selected from Gad m 1, Gad m 2, and Gad m 3. In some embodiments, a food composition comprises an allergenic protein, wherein the allergenic protein is selected from Sal s 1, Sal s 3, Sal s 2, or One k 5. In some embodiments, a food composition comprises an allergenic protein, wherein the allergenic protein is selected from Met e 1, Pan s 1, or Hom a 1. In some embodiments, a food composition comprises an allergenic protein, wherein the allergenic protein is avenin.

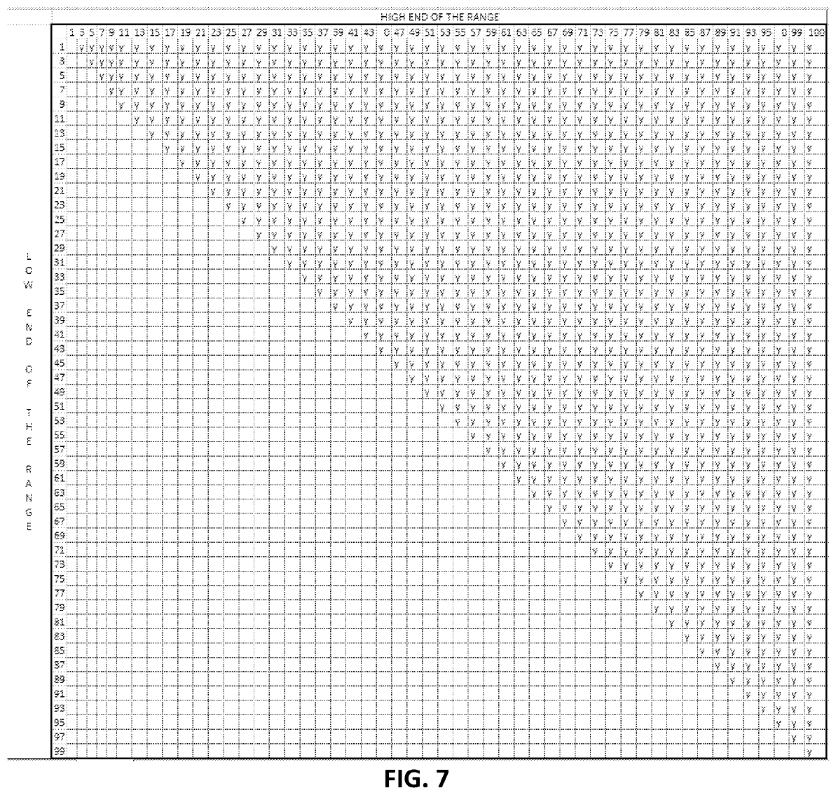

FIG. 7 provides, for some embodiments, a selection point “y” establishing the high and low ends of a range of an amount by weight of a given protein in a portion of a food composition, where a given protein has some degree of unfolding “x” (e.g., a range selected from FIG. 6), relative to the total amount, by weight, of protein in that portion of the food composition.

Thus, using the herein described techniques, a designed or “custom-tailored” food composition having one or more proteins providing varying, precise, and controllable degrees of: (1) percent unfolding of a given protein (e.g. typically correlating to a reduced number of epitopes of a particular given protein (for example, a protein selected from the proteins described herein, such as those specified in right hand column of Table 1.); and, (2) percent by weight inclusion of that given protein in a food composition, can be created.

In one such embodiment, a food composition includes a first allergenic protein where a percent range of the total number of weak bonds in the first allergenic protein are permanently broken (e.g., unfolded) and where that first allergenic protein may optionally maintain or have one or more T-cell epitopes. The percent range of broken weak bonds compared to all of the weak bonds in the first allergenic protein is a selected x position in FIG. 6. The weight percent range of that first allergenic protein relative to all of the protein in the food composition, by weight, is a determined, or selected, y position in FIG. 7, thereby creating the custom food composition.

In another embodiment, the custom food composition includes a second allergenic protein, where a percent range of the total number of weak bonds in the second allergenic protein are permanently broken (e.g., unfolded) and where that second allergenic protein may optionally maintain or have one or more T-cell epitopes. The percent range of broken weak bonds compared to all of the weak bonds in the second allergenic protein is a selected x position in FIG. 6. The percent range of broken weak bonds may be the same or different than the range of the first allergenic protein. Similarly, the weight percent range of the second allergenic protein relative to all of the protein in the food composition, including the first allergenic protein, can also be a selected y position in FIG. 7, thereby creating a custom food composition.

A food composition of the present invention can include a third, fourth, fifth, sixth, seventh or more allergenic proteins. Such proteins can, for example, be selected from the proteins described herein, such as those specified in the right hand column of Table 1.

First and second allergenic proteins can also be differentiated by degrees of percent unfolding of a given protein (e.g. typically correlating to a reduced number of epitopes of a given protein). Accordingly, in some embodiments the first and second allergenic protein can be the same protein type. For example, Cor a 9 can be used for both the first and second allergenic proteins, yet as between those same protein types, each can have differing degrees of percent unfolding. Alternatively, the first and second allergenic proteins can be different protein types each with particular degrees of unfolding, for example Gal d 2 and Cor a 9.

The first and second, and/or additional allergenic proteins can be in the same for different portions of the custom-tailored food composition.

Similarly, it follows that in some embodiments, a protein may be partially unfolded using one or more of the treatments described above, and subsequently mixed with natively-folded proteins to produce a mixture. For example, a composition comprising a natively-folded protein may be treated using heat treatment, instant controlled pressure drop, pepsination, hydrolyzation, or acid or base treatment, resulting in partial unfolding of that protein. The partially unfolded protein may then be mixed with natively-folded protein to produce a mixture.

In some embodiments, the food composition comprises fat. In some embodiments, the food composition comprises added fat, in addition to fat naturally present therein. The fat may comprise triglycerides and/or cholesterol. In some embodiments, the fat is saturated fat. In some embodiments, the fat is unsaturated fat, such as monounsaturated fat or polyunsaturated fat. In some embodiments, the fat comprises omega-3 fatty acids. In some embodiments, the food composition comprises one or more oils, such as canola, avocado, sunflower, corn, olive, peanut, walnut, coconut, safflower, soy, flaxseed, sesame, or vegetable oil.

In some embodiments, the food composition comprises fiber. In some embodiments, the food composition comprises added fiber, in addition to fiber naturally present therein. Fiber may include portions of plant-derived (or other) foods that cannot be completely broken down by human digestive enzymes. In some embodiments, the fiber is soluble fiber. In some embodiments, the fiber is insoluble fiber. In some embodiments, the fiber is derived from fruit, vegetables, beans (legumes), whole grains, or meat. In some embodiments, the fiber comprises beta-glucans (e.g., cellulose, chitin), hemicellulose (e.g., hexose, pentose), lignin, xanthan gum, resistant starch, arabinoxylan, fructans (e.g., inulin), polyuronide (e.g., pectin), alginic acid (e.g., sodium alginate, potassium alginate, ammonium alginate, calcium alginate, propylene glycol alginate, agar, carrageen), raffinose, or polydextrose.

In some embodiments, the food composition comprises one or more vitamins. Vitamins may include, for example, vitamin A, vitamin C, vitamin D, vitamin K, or vitamin E. In some embodiments, the food composition comprises a B vitamin, such as thiamine, riboflavin, niacin, pantothenic acid, biotin, vitamin B₆, vitamin B₁₂, or folate.

In some embodiments, the food composition comprises one or more minerals. For example, in some embodiments, the food composition comprises calcium, magnesium, potassium, or sodium.

The food composition may comprise one or more fats, fiber, and/or vitamins. In some embodiments, the food composition comprises one or more fats and one or more fibers. In some embodiments, the food comprises one or more fats and one or more vitamins. In some embodiments, the food composition comprises one or more fibers and one or more vitamins. In some embodiments, the food composition comprises fats, fiber, and vitamins.

In some embodiments, the food composition comprises butyrate. Butyrate is a short-chain fatty acid, typically produced by the intestinal microbiota. Butyrate is capable of increasing the production of Treg cells. Without being bound by any theory, it is believed that a sufficient presence of butyrate in the gut promotes tolerance of one or more allergenic proteins.

In some embodiments, a food composition comprising an allergenic protein may further comprise one or more non-allergenic proteins. For example, in some embodiments, the food composition may comprise one, two, three, four, five, six, seven, eight, nine, ten or more non-allergenic proteins.

In some embodiments, the food composition comprises at least one probiotic. Probiotics include live bacteria and yeast that promote gut health. Illustrative probiotic bacteria include, but are not limited to, species of Lactobacillus, Bifidobacteria, Saccharomyces, Streptococcus, Enterococcus, Escherichia, and Bacillus. In some embodiments a food composition comprises Lactobacillus. In some embodiments, a food composition comprises Bifidobacterium. In some embodiments, a food composition comprises Lactobacillus and Bifidobacterium.

In some embodiments, a food comprising an allergenic protein may also comprise a prebiotic. Prebiotics are compounds in foods that induce the growth or activity of beneficial microorganisms such as bacteria and fungi. Prebiotics can alter the composition of organisms in the gut microbiome. Illustrative prebiotics that may be used in the food compositions described herein include oligosaccharides, galactooligosaccharides, fructooligosaccharides, oligosaccharides, inulin, or a mixture thereof. The oligosaccharides may be isolated from a plant, such as chicory root, Jerusalem artichoke, onions (including leeks and garlic), legumes, wheat, asparagus, and jicama. In some embodiments, the oligosaccharides may be food additives such as inulin or oligofructose.

In some embodiments, the food compositions described herein may be nutritional or dietary supplements. The food compositions described herein may be produced in many different forms. For example, the food compositions described herein may be powders, purees, pastes, or beverages (e.g., formula). In some embodiments, the food compositions are puffs, crackers, or biscuits.

In some embodiments, the food compositions described herein may be shelf-stable for at least 3 months, at least 6 months, at least 9 months, at least 12 months, or more. In some embodiments, the food compositions are shelf-stable at room temperature. In some embodiments, the food compositions are shelf-stable at 4° C. In some embodiments, the food compositions are stable when frozen.

Methods for Treating and/or Preventing Food Allergies

The food compositions described herein may be used to treat and/or prevent food allergies. In some embodiments, a method for treating and/or preventing food allergy in a subject in need thereof comprises administering to the subject one or more of the food compositions described herein. In some embodiments, the food composition is administered once to the subject. In some embodiments, the food composition is administered multiple times to the subject, at effective intervals. In some embodiments, the food composition is administered once per day. In some embodiments, the food composition is administered multiple times per day. In some embodiments, the food composition is administered once per week. In some embodiments, the food composition is administered multiple times per week. In some embodiments, the food composition is administered once per month. In some embodiments, the food composition is administered multiple times per month.

In some embodiments, the subject is an adult subject. In some embodiments, the subject is a pediatric subject. For example, the subject may be 18 months of age or less, 12 months of age or less, or 6 months of age or less.

In some embodiments, the food composition is administered orally. The food composition may be eaten alone, or may be mixed with and/or combined with another food composition. For example, a powdered food composition described herein may be mixed with a “carrier” food for ease of administration. Suitable carrier foods may include, for example, yogurt, apple sauce, soup, oatmeal, etc.

### Kits

Also provided herein are kits comprising one or more of the food compositions described herein. The kits may comprise, for example, one or more vessels containing the food composition(s) and, optionally, packaging and instructions for use. The instructions may, for example, allow for a consumer to determine how much of the food composition to utilize based on the desired protein content per serving. Kits typically include a label indicating the intended use of the contents of the kit.

In some embodiments, the kit comprises a vessel comprising a food composition. In some embodiments, the vessel comprises a single dose of the food composition. In some embodiments, the vessel comprises multiple doses of the food composition. In some embodiments, the vessel further comprises a second food composition comprising a second allergenic protein.

In some embodiments, the kit comprises a second vessel. The second vessel may comprise, in some embodiments, a second allergenic protein. In some embodiments, the kit comprises a first vessel and second vessel, wherein the amount of partially denatured allergenic protein in the first vessel is different from the amount of partially denatured allergenic protein in the second vessel. For example, the amount of partially denatured allergenic protein in the first vessel may be greater or smaller than the amount in the second vessel. In some embodiments, the kit comprises a first vessel and second vessel, wherein the amount of partially denatured allergenic protein in the first vessel is the same as the amount of partially denatured allergenic protein in the second vessel.

In some embodiments, the kit comprises a first, second, third, and fourth vessel, wherein the amount of partially denatured allergenic protein is different in each vessel. For example, the amount of partially denatured allergenic protein may be greater in the second vessel than the first, greater in the third vessel than the second, and/or greater in the fourth vessel than the third. In some embodiments, the amount of partially denatured allergenic protein may be less in the fourth vessel than the third, less in the third vessel than the second, and/or less in the second vessel than the first. In some embodiments, the kit comprises a first, second, third, and fourth vessel, wherein the amount of partially denatured allergenic protein is the same in each vessel.

The vessels comprising the food composition may have any size and/or configuration. For example, in some embodiments, the vessel is a sachet, bag, or tube. In some embodiments, the vessel is a spoon, such as a preloaded spoon.

### Incorporation by Reference

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

### EXAMPLES

The disclosure is further illustrated by the following examples, which are not to be construed as limiting this disclosure in scope or spirit to the specific procedures described in the disclosure. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure.

### Example 1: Production of Food Compositions Comprising Partially Unfolded Allergenic Proteins from Milk and Egg

The instant inventors hypothesized that foods containing a mix of naturally folded and T-cell epitope-containing proteins can optimally balance reduced immunogenicity with adequate antigenic properties. Furthermore, by preserving the food in as natural form as possible, an overall tolerance training effect could be achieved. Accordingly, the instant inventors sought to prepare milk and egg compositions comprising varying levels of partially denatured allergenic proteins, while also retaining some of the allergenic protein in a natively folded configuration.

The inventors used heat treatment including toasting, boiling, baking and combinations thereof to partially denature the allergenic proteins. It was hypothesized that the length of the heat treatment would affect the extent to which the foods were denatured. It was further hypothesized that the specific mechanism of heating (e.g., boiling vs. baking) would also be important.

Milk and egg, containing known allergenic proteins, were subjected to various different types of heat treatment, for different amounts of time. For example, samples were toasted for about 0, 5, or 10 minutes; boiled for about 0, 10, 20, or 30 minutes; baked for about 0, 10, 20, or 30 minutes; or boiled for 10 minutes followed by 5 or 10 minutes of toasting. In this example, the toasting methods were performed by heating a spray-dried version of the milk or egg using a heat source placed adjacent thereto, wherein the heat source was at a temperature of about 167° C. Boiling was performed by heating wet milk or egg compositions alone or in water, maintained at a temperature of about 100° C. Baking involved mixing a wet form of the milk or egg compositions in a 1:1 ratio with a starch (cassava flour or tapioca starch). The mixture was then placed in an oven at a temperature of about 167° C., which caused the food to rise to the necessary temperature without burning. After heat treatment, levels of reactive allergenic proteins were measured using a standard ELISA.

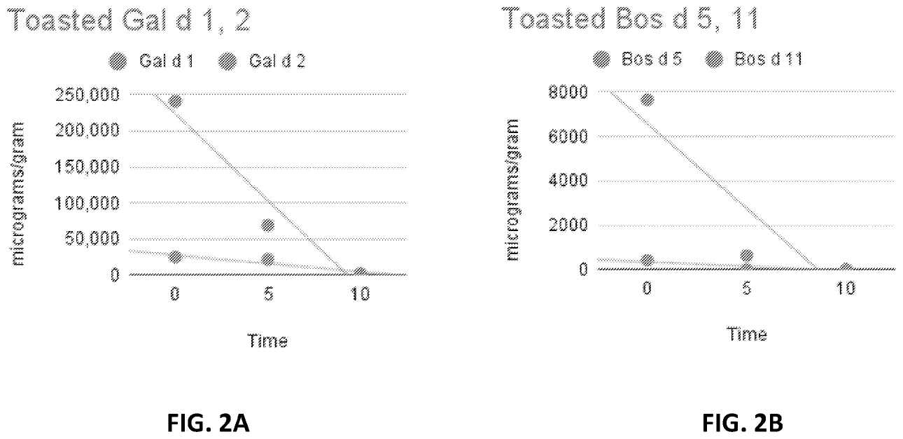

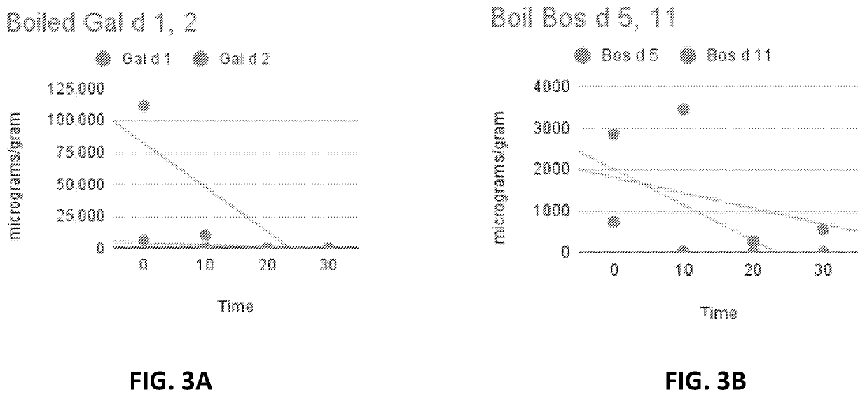

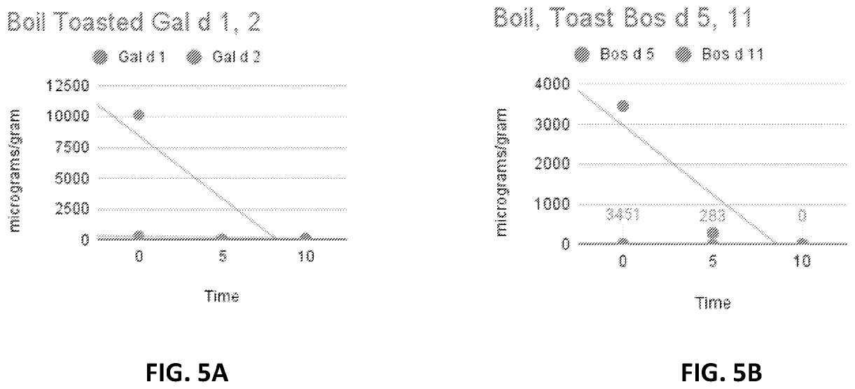

As shown in Table 1 and in FIG. 2B, toasting for 5 minutes reduced levels of reactive Bos d 5 and Bos d 11 compared to the untreated milk sample. After 10 minutes of toasting, no reactive Bos d 5 and Bos d 11 were detected. Boiling and baking the samples for longer periods of time substantially reduced, but did not eliminate reactive Bos d 5 and Bos d 11 (FIG. 3B). In samples that were boiled for 10 minutes followed by toasting, no reactive Bos d 5 was detected (FIG. 5B). However, reactive Bos d 11 was still detected following 10 minutes of boiling plus 5 minutes of toasting.

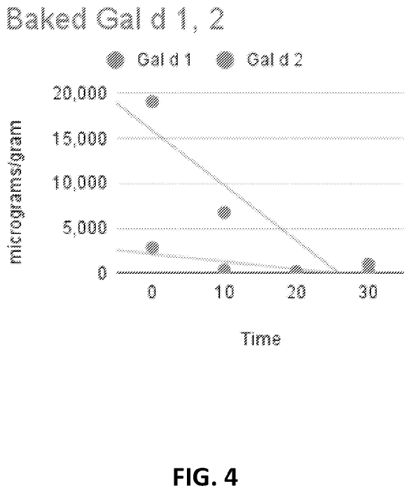

As shown in Table 2, FIG. 2A, and FIG. 3A, toasting for 5 or 10 minutes or boiling for 10, 20, or 30 minutes reduced but did not eliminate reactive Gal d 1 and Gal d 2. Baking for 0, 10, 20, or 30 minutes did not eliminate reactive Gal d 1, but baking for 30 minutes did eliminate—reactive Gal d 2 (FIG. 4). Detectable levels of reactive Gal d 1 and Gal d 2 were also maintained after boiling for 10 minutes and toasting for 5 or 10 minutes (FIG. 5A).

TABLE 1
Levels of reactive Bos d 5 and Bos d 11 in heat-treated milk

| | | reactive Bos d 5 | | | |
|---|---|---|---|---|---|
| | | (percent | | reactive Bos d 11 | |
| | | reduction | | (percent reduction | |
| Method | Time | relative to t = 0) | | relative to t = 0) | |
| Toast | 0 | 421 | | 7651 | |
| | 5 | 5 | (>99%) | 634 | (92%) |
| | 10 | 0 | (100%) | 0 | (100%) |
| Boil | 0 | 2854 | | 728 | |
| | 10 | 14 | (>99%) | 3451 | |
| | 20 | 2 | (>99%) | 264 | (64%) |
| | 30 | 1 | (>99%) | 548 | (25%) |
| Boil 10 | 5 | 0 | | 283 | |
| Toast | 10 | 0 | (100%) | 0 | (100%) |

TABLE 2
Levels of reactive Bos d 5 and Bos d 11 in heat-treated milk

| | | reactive Gal | reactive Gal |
|---|---|---|---|
| | | d 1 (percent | d 2 (percent |
| | | reduction | reduction |
| | | relative to | relative to |
| Method | Time | t = 0) | t = 0) |
| Toast | 0 | 24,946 | 240,844 |

| | | | | |
|---|---|---|---|---|
| 5 | 21,745 | (13%) | 69,197 | (71%) |
| 10 | 1,986 | (84%) | 943 | (>99%) |

| | | | |
|---|---|---|---|
| Boil | 0 | 6,235 | 111,596 |

| | | | | |
|---|---|---|---|---|
| 10 | 326 | (95%) | 10,145 | (91%) |
| 20 | 117 | (98%) | 25 | (>99%) |
| 30 | 21 | (>99%) | 61 | (>99%) |

| | | | |
|---|---|---|---|
| Bake | 0 | 2,861 | 19,088 |

| | | | | |
|---|---|---|---|---|
| 10 | 362 | (87%) | 6,773 | (65%) |

| | | | |
|---|---|---|---|
| 20 | 202 | (93%) | 1,784 |
| 30 | 112 | (96%) | 0 |

| | | | |
|---|---|---|---|
| Boil 10 | 5 | 95 | 0.25 |
| Toast | 10 | 157 | 2.3 |

Taken together, these data indicate that the tested methods of heat treatment may be used to partially denature allergenic proteins from milk and egg, to a controllable extent. It also shows how different heating methods/time periods of treatment may be used to reduce or eliminate the reactivity of selective proteins, as compared to other allergenic proteins in the same original composition. For example, in these experiments, the methods involving lower heat levels (i.e., boiling) seemed to preferentially denature one of the proteins while protecting the other. Notably, the combination of boiling and toasting of a wet starting composition eliminated the need to use starch.

### Example 2: Methods of Unfolding Peanut Proteins to Produce Compositions with Lower Allergenicity

Peanut flour was diluted to a 20% slurry using high purity deionized water (18 MΩ) and then heated until a reflux or bubbling was observed, which was considered the boiling time start point. Boiling proceeded for 60 min, with sampling occurring at 5, 10, 15, 30, 45, and 60 min intervals. The slurries were covered with a watch glass and aluminum foil except during sampling. For low pH conditions, 0.1 M sulfuric acid was used to achieve a starting pH of 3 and, for high pH conditions, 0.03 M sodium hydroxide was used to achieve a starting pH of 8. Samples were freeze dried prior to MARIA testing.

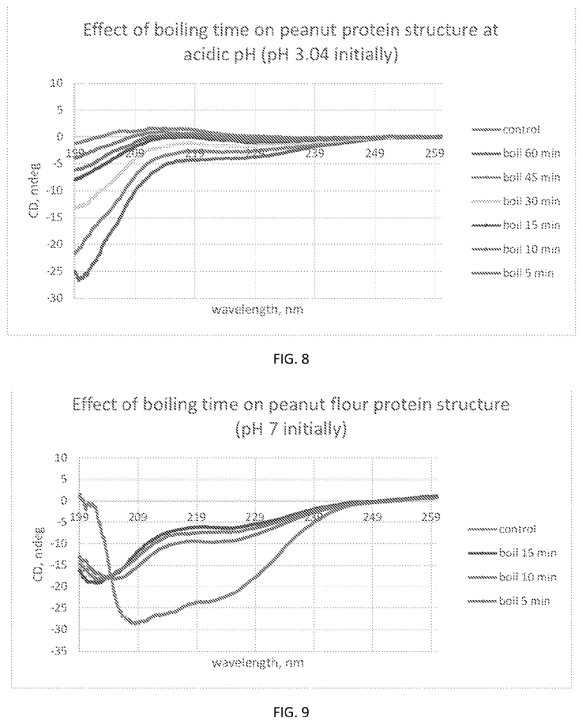

The effect of the treatment on the peanut protein structure was evaluated by circular dichromism. Results are shown in FIG. 8 for low pH conditions, FIG. 9 for neutral conditions and FIG. 10 for high pH conditions.

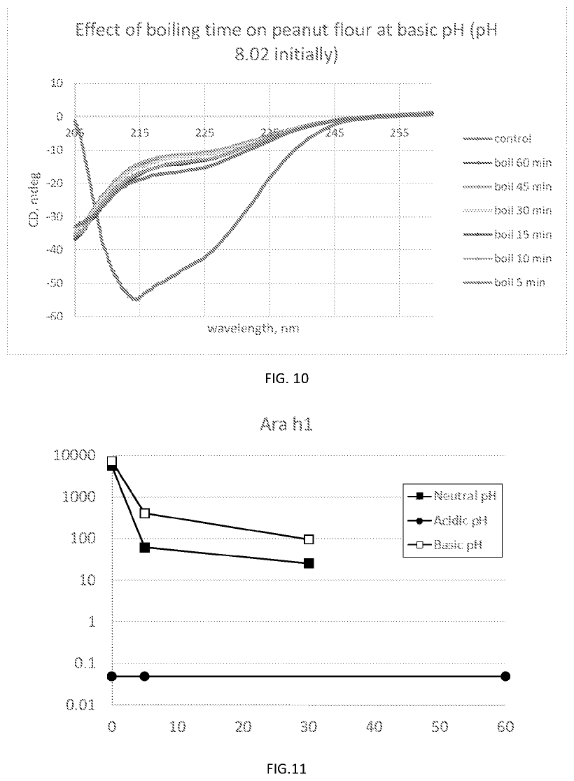

Table 3 and FIGS. 11, 12 and 13 summarize the impact of pH and boiling duration on protein allergenicity measured with the proteins Ara h 1, Ara h 3, and Ara h 6 for peanut.

TABLE 3
Allergenicity of peanut proteins after pH-boiling
treatments using MARIA assay in μg/g

| Code | Description | Ara h 1 | Ara h 3 | Ara h 6 |
|---|---|---|---|---|
| PFC-1 | control | 5,375.35 | 24,007.00 | 5,439.71 |
| PFC-2 | control | 5,874.48 | 22,399.50 | 5,798.27 |
| | boil 5 min, neutral | | | |
| E | pH | 61.72 | 3,628.76 | 4,349.65 |
| | boil 30 min, neutral | | | |
| A | pH | 30.63 | 97.21 | 2,356.64 |
| | boil 30 min, neutral | | | |
| G | pH | 20.25 | 573.28 | 3,286.64 |
| PFAC | control, acidic pH | <0.049 | 0.38 | 2,907.57 |
| K | boil 5 min, acidic pH | <0.049 | <0.024 | 3,348.37 |
| | boil 60 min, acidic | | | |
| P | pH | <0.049 | <0.024 | 2,542.55 |
| PFBC | control, basic pH | 7,355.20 | 51,124.50 | 6,127.56 |
| AU | boil 5 min, basic pH | 407.11 | 4,935.23 | 3,328.37 |
| | boil 30 min, basic | | | |
| AX | pH | 96.06 | 866.81 | 302.41 |

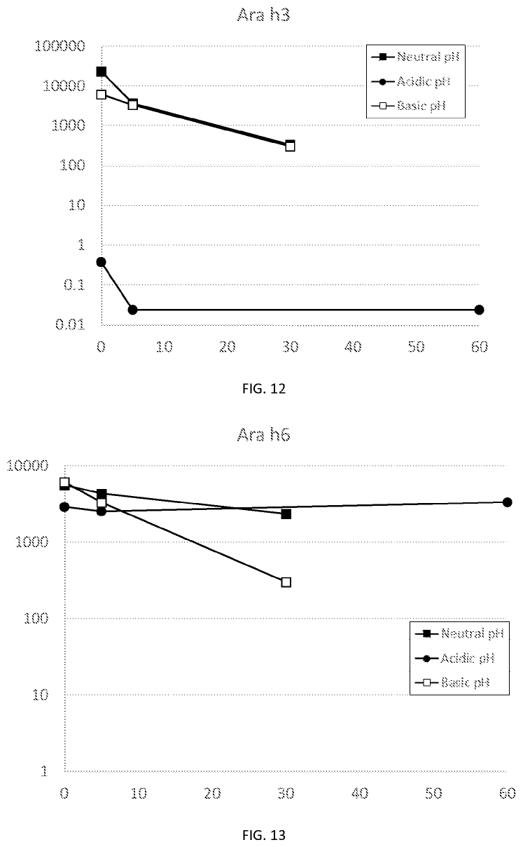

As observed in Table 3 and FIGS. 11 and 12, drastic reduction in the allergenicity of Ara h 1 and 3 is possible with a short boiling time (5 min) and without any pH adjustment. Further, with acid pH adjustment (pH 3), the antibody response to Ara h 1 and 3 is less than 0.4 μg/g, even without heat treatment. Boiling under acidic conditions for only 5 min is able to reduce Ara h 1 and 3 to below the test detection limit. In contrast as shown in FIG. 13, Ara h 6 requires 30 min of boiling without pH adjustment to obtain a 50% reduction allergenicity. Boiling under acidic conditions for 60 min provides no further reduction to Ara h 6 allergenicity. Ara h 6 has been found to be protease resistant and heat stable, which can be explained by the 6 disulphide bonds in its core.

Interestingly, basic conditions before boiling increase the allergenicity of all three tested allergens. Yet, boiling for 30 min under basic conditions was most successful at reducing Ara h 6 allergenicity, resulting in about 5% of the original level. These conditions also reduced Ara h 1 and 3 allergenicity to less than 5% of the original level. Boiling peanut flour under basic conditions for ≥30 min is a preferred condition for reducing allergenicity.

### Example 3: Methods of Unfolding Almond Proteins to Produce Compositions with Lower Allergenicity

Almond flour was diluted to a 30% slurry using high purity deionized water (18 MΩ) and then heated until a reflux or bubbling was observed, which was considered the boiling time start point. Boiling proceeded for 60 min, with sampling occurring at 5, 10, 15, 30, 45, and 60 min intervals. The slurries were covered with a watch glass and aluminum foil except during sampling. For low pH conditions, 0.1 M sulfuric acid was used to achieve a starting pH of 3 and, for high pH conditions, 0.03 M sodium hydroxide was used to achieve a starting pH of 8. Samples were freeze dried prior to MARIA testing.

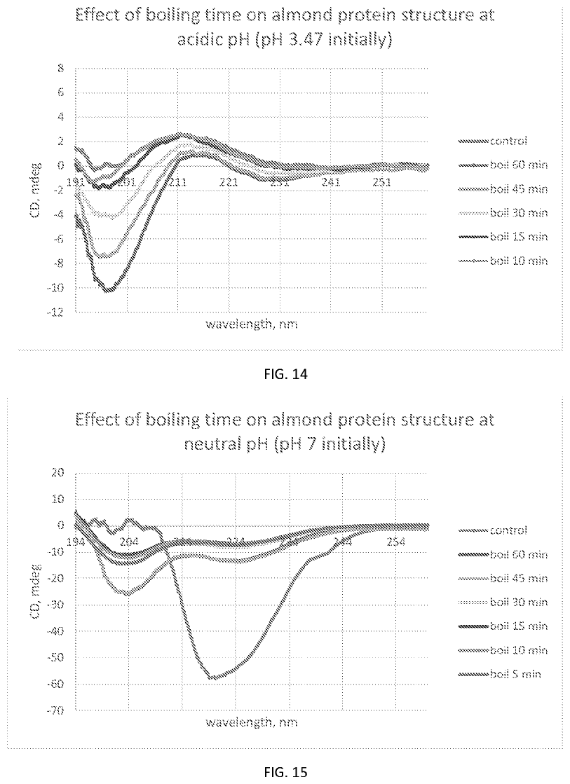

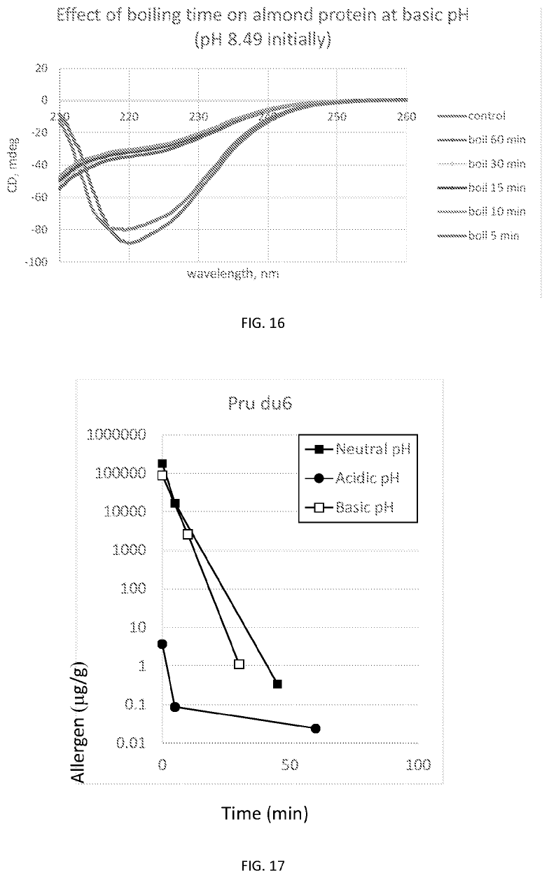

The effect of the treatment on the almond protein structure was evaluated by circular dichromism. Results are shown in FIG. 14 for low pH conditions, FIG. 15 for neutral conditions and FIG. 16 for high pH conditions.

Table 4 and FIG. 17 summarize the impact of pH and boiling duration on protein allergenicity measured with the proteins Pru du 6 for almond. Almond allergenicity was tested using Pru du 6. All boiling conditions drastically reduced allergenicity. Under neutral conditions, at least 5 min of boiling is needed. The two replicates of 5 min boiling show large variation, so a longer time would be preferred to ensure sufficient time for heat transfer. As observed with peanut, a pH change to pH 3 without heat is sufficient to reduce most of the allergenic response to Pru du 6. Boiling under basic conditions is also effective at reducing Pru du 6 allergenicity but the same result is achieved with less time under acidic conditions.

TABLE 4
Allergenicity of almond proteins after pH-boiling
treatments using MARIA assay in μg/g

| Code | Description | Pru du 6 |
|---|---|---|
| APC-1 | control | 175,080.00 |
| APC-2 | control | 184,730.00 |
| Q | boil 5 min, neutral pH | 0.74 |
| W | boil 5 min, neutral pH | 33,602.50 |
| U | boil 45 min, neutral pH | 0.54 |
| AA | boil 45 min, neutral pH | 0.14 |
| APAC | control, acidic pH | 3.71 |
| Al | boil 5 min, acidic pH | 0.09 |
| AN | boil 60 min, acidic pH | <0.024 |
| APBC | control, basic pH | 87,880.00 |
| AP | boil 10 min, basic pH | 2,628.29 |
| AR | boil 30 min, basic pH | 1.12 |

1. A food composition comprising a first portion of an allergenic protein;
wherein between 1% and 99% of the allergenic protein in the composition is partially unfolded, wherein the partially unfolded allergenic protein comprises one or more T-cell epitopes and is substantially free of IgE epitopes and/or B-cell epitopes; and
wherein at least 1% of the allergenic protein in the composition is natively folded.

2. The food composition of claim 1, wherein between 75% and 99% of the allergenic protein is partially unfolded.

3. The food composition of claim 1, wherein the allergenic protein comprises at least one selected from the following:
(a) a peanut allergenic protein selected from Ara h 1, Ara h 2, Ara h 3, Ara h 6, or Ara h 9;
(b) a walnut allergenic protein selected from Jug r1 or Jug r3;
(c) a pistachio allergenic protein selected from Pis v 1, Pis v 2, Pis v 3, or Pis V 4;
(d) a cashew allergenic protein selected from AnaO3;
(e) a hazelnut allergenic protein selected from Cor a 9, Cor a 14, Cor a 1, or Cor a 8;
(f) a pecan allergenic protein selected from Car i 1;
(g) an almond allergenic protein selected from Pru du 1, 2, 3, 4, 5, 6, or 8;
(h) a brazil nut allergenic protein selected from Ber e1 or e2;
(i) a wheat allergenic protein selected from wheat Tri a 19 or Tri a 14;
(j) a soy allergenic protein selected from Gly m Bd 60K, Gly m Bd 30K, or Gly m Bd 28K;
(k) a pea allergenic protein selected from Pis s 1 or Pis s 2;
(l) a cod allergenic protein selected from Gad m 1, Gad m 2, or Gad m 3;
(m) a salmon allergenic protein selected from Sal s 1, Sal s 3, Sal s 2, or Onc k 5;
(n) a shrimp allergenic protein selected from Met e 1, Pan s 1, or Hom a 1; or
(o) an oat allergenic protein selected from avenin.

4. The food composition of claim 3, comprising at least one additive selected from the following: fat, fiber, one or more vitamins, butyrate, one or more non-allergenic proteins, or at least one probiotic, wherein the probiotic is optionally selected from lactobacillus or bifidobacterium.

5. The food composition of claim 1, wherein the composition comprises a prebiotic.

6. The food composition of m claim 1, wherein the composition is a powder, a puree, a paste, or a beverage.

7. The food composition of m claim 1, wherein the composition is a puff, cracker, or biscuit.

8. A method of partially unfolding an allergenic protein in a food composition, the method comprising treating the food composition using the appropriate treatment for a given protein selected from:
i) heat treatment;
ii) instant controlled pressure drop;
iii) pepsination;
iv) hydrolyzation;
v) acid treatment; or
vi) fermentation
wherein the treatment reduces the amount of natively folded allergenic protein in the food composition by at least 50%; and
wherein the partially unfolded allergenic protein comprises one or more T-cell epitopes and is substantially free of IgE epitopes and/or B-cell epitopes.

9. The method of claim 8, wherein the treatment reduces the amount of natively folded allergenic protein in the food composition, wherein the amount of unfolded protein, by weight, is a range “y” selected from FIG. 7, with the remainder of the amount of protein, by weight, is natively folded allergenic protein.

10. The method of claim 8, wherein the treatment reduces the amount of natively folded allergenic food protein in the food composition by at least 90%.

11. A method of treating and/or preventing food allergy in a subject in need thereof, the method comprising administering to the subject the food composition of claim 1.

12. The food composition of claim 1, wherein about 1% of a total number of weak bonds in the allergenic protein first portion are permanently broken.

13. The food composition of claim 1, wherein about 75% of a total number of weak bonds in the allergenic protein first portion are permanently broken.

14. The food composition of claim 1, wherein about 90% of a total number of weak bonds in the allergenic protein first portion are permanently broken.

15. A kit comprising a vessel comprising the food composition of claim 1.

16. The kit of claim 15, wherein the vessel further comprises a second food composition comprising a second allergenic protein.

17. The kit of claim 15, wherein the kit comprises a second vessel comprising a second allergenic protein.

18. The kit of claim 15, wherein the kit comprises a first vessel and second vessel, wherein the amount of partially denatured allergenic protein in the first vessel is different from the amount of partially denatured allergenic protein in the second vessel.

19. The food composition of claim 1, further comprising a second portion of a second allergenic protein; wherein the second allergenic protein may be the same or different than the allergenic protein of the first portion, and between 0% and 100% of the total number of weak bonds in the second allergenic protein of the second portion are permanently broken.

20. A food composition comprising a first allergenic protein; wherein a percent range of the total number of weak bonds in the first allergenic protein are permanently broken, wherein the first allergenic protein maintains one or more T-cell epitopes and the unfolded part of the protein is substantially free of IgE epitopes and/or B-cell epitopes relative to a native structure of the protein, wherein that percent range of broken weak bonds compared to all of the weak bonds in the first allergenic protein is a selected x position in FIG. 1, and wherein a percent range of the amount by weight of the first allergenic protein relative to all of the protein in the food composition by weight is a selected y position in FIG. 2.

21. The method of claim 8, wherein the heat treatment comprises an acidic pH, a neutral pH or a basic pH.
